(19) United States
(12) Patent Application Publication
(10) Pub. No.: US 2008/0220432 A1
(43) Pub. Date: Sep. 11, 2008

(54) Optimized host cells for protein production

(75) Inventors: Dennis Sawchuk, Hoboken, NJ (US); Kambiz Shekdar, New York, NY (US)

Correspondence Address: ROPES & GRAY LLP, PATENT DOCKETING 39/361, 1211 AVENUE OF THE AMERICAS, NEW YORK, NY, 10036-8704, US

(73) Assignee: CHROMOCELL CORPORATION, North Brunswick, NJ (US)

(21) Appl. No.: 11/998,646

(22) Filed: Nov. 30, 2007

Related U.S. Application Data
(60) Provisional application No. 60/872,281, filed on Nov. 30, 2006.

(51) Int. Cl. C12Q 1/68; C12Q 1/04

(52) U.S. Cl. 435/6; 435/34

(57) ABSTRACT

The present invention relates to methods for isolating cells that express increased levels of an RNA or protein of interest, wherein the cells exhibit altered growth profiles, such as cells with increased or decreased rates of proliferation, increased or decreased rates of apoptosis, or cells with a biphasic growth profile.

### CROSS-REFERENCE TO RELATED APPLICATIONS

This application claims the benefit of priority from U.S. Provisional Application No. 60/872,281, filed Nov. 30, 2006. The contents of this application are incorporated by reference herein in their entirety.

### BACKGROUND OF THE INVENTION

Selection of cell lines that produce high levels of recombinant protein is one of the greatest challenges in biotechnology. Host populations of cells used for protein production often represent genetically heterogeneous cells having different growth and other properties. Even when such host populations represent populations derived from an individual cell, the culture of such a cell over time results in population of cells in which individual cells may be characterized by one or more accumulated genetic differences. For protein production, one or more genetic sequences encoding the protein(s) of interest are introduced into the cells. Each cell of the population introduced with the genetic sequence(s) may uptake a variable copy number of the genetic sequences and each of these may integrate in a variable position within the genome of that cell. As a result, a great diversity of cells results where each cell may have a different potential for the production of the protein of interest. For protein production, it is possible to use either all or a portion of the host cells introduced with the genetic sequences, or it is possible to test clonal populations of cells, or cell lines, derived from individually isolated cells from this population to identify those with optimal protein production and other characteristics such as certain growth or proliferation characteristics that may be beneficial for protein production. Given the great diversity of cells, it is difficult to identify and isolate an individual cell with the capacity for increased production of an RNA of interest (e.g., an RNA that encodes a protein of interest) in a population of thousands or millions of cells. Limiting dilution is one common method for the identification of cell lines for protein production where several hundred and if automated by robotics several thousand individually isolated cells are cultured to give rise to clonal populations which are then assessed for protein production. However, due to the great diversity of cells with respect to both protein production and other characteristics such as growth or proliferation rates, testing such a relatively small number of cells is an inefficient method of identifying the most optimal cell lines. Flow cytometry or cell sorting, with its ability to analyze and separate single cells, is another method that may aid in the selection of such rare cells by enabling a greater number of individual cells to be tested. However, many standard methods used in flow cytometry for measuring RNA or protein production often require killing the cells that are being measured or are unable to measure protein production in individual cells. In addition, these methods as currently applied do not allow an assessment of the proliferation rates of cells. Accordingly, there is a need for methods for high-throughput methods for identifying, isolating, and cultivating cells with increased rates of RNA or protein production where the cells are also selected according to optimized growth and proliferation properties.

Previous methods for increasing protein production in cells have also focused on the optimization of media formulations. For example, various components of growth media such as sugars, salts, amino acids, vitamins, etc. were increased or decreased. See, for example, Chu and Robinson, Curr Opin Biotechnol. 2001 April; 12(2):180-7; Chun et al., Biotechnol Prog 2003 January-February; 19(1):52-7; Dempsey et al., Biotechnol Prog 2003 January-February; 19(1):175-8; and Sauer et al., Biotechnol Bioeng. 2000 Mar. 5; 67(5):585-97. These efforts may be focused on heterogeneous populations of cells. It is also possible to generate a clonal population of cells that reliably produce high levels of protein and to optimize media for such a clonal population.

In addition, cells that express high levels of an RNA of interest may spend much of their energy on protein production and thus suffer reduced growth rates (Gu et al., Cytotechnology. 1992; 9(1-3):237-45 and Kromenaker et al., Biotechnol Prog. 1994 May-June; 10(3):299-307). In particular when a non-clonal population of cells is used, the reduced growth rates can lead to overgrowth by the cells with decreased protein production. Methods to select cells with optimal growth and proliferation profiles under different or optimized media conditions would also be helpful for establishing populations of cells for optimized protein production.

### SUMMARY OF THE INVENTION

The present invention relates to methods for isolating cells that express increased levels of an RNA or protein of interest. The invention also relates to methods for isolating cells with altered rates of cell proliferation, such as cells with increased or decreased rates of cell proliferation. Also provided are methods for isolating cells with altered rates of cell proliferation (e.g., increased or decreased rates of cell proliferation), wherein the cells also express increased levels of an RNA or protein of interest.

In one embodiment, the invention provides a method for isolating a cell with an increased rate of cell proliferation. The method comprises the steps of contacting a population of cells with a fluorescent reagent for monitoring the rate of cell proliferation and isolating the cell that exhibits a level of fluorescence of the fluorescent reagent that correlates with increased cell proliferation.

In another embodiment, the invention provides a method for isolating a cell with an increased rate of cell proliferation, wherein the cell also expresses high levels of an RNA of interest. The method comprises the steps of contacting a population of cells with a fluorogenic probe that fluoresces upon hybridization to the RNA of interest; contacting the population with a fluorescent reagent for monitoring the rate of cell proliferation; and isolating the cell that exhibits increased fluorescence of the fluorogenic probe and a level of fluorescence of the fluorescent reagent that correlates with increased cell proliferation. The detection of the fluorescence of the fluorogenic probe can be assayed simultaneously with detection of the fluorescence of the reagent for monitoring the rate of cell proliferation. Alternatively, the detection of the fluorescence of the fluorogenic probe can be assayed in a separate step than detection of the fluorescence of the reagent for monitoring the rate of cell proliferation. The fluorescent reagent for monitoring the rate of cell proliferation may fluoresce at the same or different wavelength than that of the fluorogenic probe. A cell isolated according a method of the invention may further be cultured to produce a cell culture or cell line. In certain embodiments, the above method further comprises the step of measuring the density of the cell culture.

In another embodiment, the invention provides a method for producing a cell culture with increased cell density, wherein cells in the cell culture express increased levels of an RNA of interest. The method comprises the steps of contacting a population of cells with a fluorogenic probe that fluoresces upon hybridization to the RNA of interest; isolating a cell from the population that exhibits increased fluorescence of the fluorogenic probe; culturing the isolated cell to produce a first cell culture; repeating the previous steps to isolate a second cell culture; measuring the density of the first and second cell cultures; and identifying the cell culture with increased or higher cell density wherein cells in the cell culture express increased high levels of the RNA of interest.

In certain embodiments, the invention also provides a method for isolating a cell with a biphasic growth profile, wherein the cell has an increased rate of proliferation in the first portion of the growth profile, and wherein the cell has a decreased rate of proliferation in the second portion of the growth profile. In one embodiment, the method comprises the steps of contacting a population of cells with a fluorescent reagent for monitoring the rate of cell proliferation and isolating the cell that exhibits altered fluorescence of the fluorescent reagent in the first portion of the growth profile and unaltered or reduced fluorescence of the fluorescent reagent in the second portion of the growth profile.

In certain other embodiments, the invention provides a method for isolating a cell with a biphasic growth profile, wherein, the cell has an increased rate of proliferation in the first portion of the growth profile, and wherein the cell has a decreased rate of proliferation in the second portion of the growth profile, and wherein the cell expresses equal or higher levels of an RNA of interest in the second portion of the growth profile than in the first portion of the growth profile. In other embodiments, the invention provides a method for isolating a cell with a biphasic growth profile, wherein, the cell has an increased rate of proliferation in the first portion of the growth profile, and wherein the cell has a decreased rate of proliferation in the second portion of the growth profile, and wherein the cell expresses lower levels of an RNA of interest in the second portion of the growth profile than in the first portion of the growth profile. In one embodiment, the method comprises the steps of: contacting a population of cells with a fluorogenic probe that fluoresces upon hybridization to said RNA of interest; contacting the population with a fluorescent reagent for monitoring the rate of cell proliferation, wherein the reagent fluoresces at a wavelength different than that of the fluorogenic probe; and isolating the cell that exhibits altered fluorescence of the fluorescent reagent in the first portion of the growth profile, unaltered or reduced fluorescence intensity change of the fluorescent reagent in the second portion of the growth profile, and increased fluorescence of the fluorogenic probe in the second portion of the growth profile. In another embodiment, the method comprises the steps of: contacting a population of cells with a fluorogenic probe that fluoresces upon hybridization to said RNA of interest; contacting the population with a fluorescent reagent for monitoring the rate of cell proliferation, wherein the reagent fluoresces at a wavelength different than that of the fluorogenic probe; and isolating the cell that exhibits altered fluorescence of the fluorescent reagent in the first portion of the growth profile, increased fluorescence of the fluorescent reagent in the second portion of the growth profile, and increased fluorescence of the fluorogenic probe in the second portion of the growth profile. In another embodiment, the method comprises the steps of: contacting a population of cells with a fluorogenic probe that fluoresces upon hybridization to said RNA of interest; contacting population with a fluorescent reagent for monitoring the rate of cell proliferation, wherein the reagent fluoresces at a wavelength different than that of the fluorogenic probe; and isolating the cell that exhibits altered fluorescence of the fluorescent reagent in the first portion of the growth profile, unaltered or reduced fluorescence of the fluorescent reagent in the second portion of the growth profile, and decreased fluorescence of the fluorogenic probe in the second portion of the growth profile. In one embodiment, the detection of the fluorescence of the fluorogenic probe is assayed simultaneously with detection of the fluorescence of the reagent for monitoring the rate of cell proliferation during the second portion of the growth profile. Alternatively, the detection of the fluorescence of the fluorogenic probe is assayed in a separate step than detection of the fluorescence of the reagent for monitoring the rate of cell proliferation. In another embodiment, the fluorescent reagent for monitoring the rate of cell proliferation fluoresces at the same wavelength as the fluorogenic probe. A cell isolated according a method of the invention may further be cultured to produce a cell culture or cell line. In certain embodiments, the above method further comprises the step of measuring the density of the cell culture.

Any of the methods described herein may further comprise contacting the cells of the invention with a reagent for monitoring an apoptotic or pre-apoptotic marker. In one embodiment, the methods described herein further comprises the step of contacting a cell with an increased rate of proliferation or increased RNA or protein production with a reagent for monitoring an apoptotic or pre-apoptotic marker. For instance, a cell that exhibits increased fluorescence of a fluorogenic signaling probe and altered fluorescence of a reagent for monitoring the rate of cell proliferation may be contacted with a reagent for monitoring an apoptotic or pre-apoptotic marker. Cells that display apoptotic or pre-apoptotic markers may be negatively selected.

RNAs that may be detected using the methods of the present invention include endogenous or heterologous RNAs that may include, without limitation, messenger RNAs that encode a protein, antisense RNA molecules, structural RNAs, ribosomal RNAs, hnRNAs, and snRNAs.

In a particular embodiment, the methods of the present invention are used to detect an mRNA that encodes an immunoglobulin heavy chain, an immunoglobulin light chain, a single chain Fv, fragments of antibodies, such as Fab, Fab′, or (Fab′)₂, or an antigen binding fragment of an immunoglobulin.

In certain embodiments of the invention, the detection of fluorescence is assayed by fluorescence microscopy, fluorocytometry, flow cytometric cell sorting technology, or by a fluorescent plate reader. The detection of fluorescence may be detected in individual samples or in multiple samples at once, such as in a high-throughput assay.

In certain embodiments of the invention, the fluorescent reagent for monitoring the rate of cell proliferation is selected from the group consisting of: carboxyfluorescein diacetate succinimidyl ester, SNARF-1 carboxylic acid, acetate succinimidyl ester, PKH26, Hoechst CPA 1, Cyquant GR and NF dyes, MTT, and CTT.

In certain embodiments, the methods of the present invention are useful for isolating and/or culturing a mammalian cell, a bacterial cell, an insect cell, a plant cell, a microbial cell, an algal cell or a fungal cell. In one embodiment, the mammalian cell is selected from the group consisting of: a Chinese Hamster Ovary (CHO) cell, a NS0 cell, a HEK 293 cell, a Per.C6 cell. In certain embodiments, the CHO cell is a CHOK1 cell, a CHOK1SV cell, a CHO-S cell, or a DG44 cell. In another embodiment, the bacterial cell is a BL21 cell. In yet another embodiment, the fungal cell is selected from the group consisting of: a Chrysosporium cell, an Aspergillus cell, a Trichoderma cell, a Dictyostelium cell, a Candida cell, a Saccharomyces cell, a Schizosaccharomyces cell and a Penicillium cell. In another embodiment, the insect cell is a SF9 cell or a SF21 cell.

### DETAILED DESCRIPTION OF THE INVENTION

### 1. Definitions

The term “adjacent” as used in the context of probes refers to a condition of proximity to allow an interacting pair to functionally interact with each other. For example, the condition of proximity allows a fluorophore to be quenched or partially quenched by a quencher moiety. The distance required for currently known fluorophore and quencher to interact is about 20-100 Å.

The term “biomass” refers to a population of two or more viable cells. The viable cells can be in any volume of culture media.

The term “bulge region” refers to a single-stranded region of one nucleotide or modified nucleotide that is not basepaired. The bulged nucleotide can be flanked by mutually complementary regions.

The term “dumbbell structure” refers to a strand of nucleic acid or modified nucleic acid having the conformation of two stem-loop structures linked via the end of an arm from each of the stem regions. The linkage may be a non-complementary region, or a phosphodiester linkage with or without modification.

The term “interacting pair” refers to two chemical groups that functionally interact when adjacent to each other, and when not adjacent to each other, produce a detectable signal compared to the absence of signal or background signal produced by the interacting chemical groups, or produce a different signal than the signal produced by the interacting chemical groups. An interacting pair includes, but is not limited to, a fluorophore and a quencher, a chemiluminescent label and a quencher or adduct, a dye dimer and FRET donor and acceptor, or a combination thereof. A signaling probe can comprise more than one interacting pair. For example, a wavelength-shifting signaling probe has a first fluorophore and a second fluorophore that both interact with the quencher, and the two fluorophores are FRET donor and acceptor pairs.

The term “loop region” refers to a single-stranded region of more than one nucleotide or modified nucleotide that is not base-paired. The loop can also be located between two regions of one or more nucleotides that are mutually complementary or partially complementary to each other. For example, the region upstream of the loop is complementary or partially complementary to the region downstream of the loop.

The term “signaling probe” refers to a probe comprising a sequence complementary to a target nucleic acid sequence and at least a mutually complementary region, and further comprising at least an interacting pair. When the signaling probe is not bound to its target sequence, the moieties of the interacting pair are adjacent to each other such that no or little or different signal is produced. When the signaling probe is bound to the target sequence, the moieties of the interacting pair are no longer adjacent to each other and a detectable signal or a different signal than the signal produced by the probe in its unbound state is produced. In one embodiment, the signaling probe is a fluorogenic probe that comprises a fluorophore and a quencher moiety, and a change in fluorescence is produced upon hybridization to the target sequence. The moieties of the interacting pair may be attached to the termini of the signaling probe or may be attached within the nucleic acid sequence. Examples of moieties that may be incorporated internally into the sequence of the signaling probe include, without limitation, the quenchers: dabcyl dT, BHQ2 dT, and BHQ1 dT, and the fluorophores: fluorescein dT, Alexa dT, and Tamra dT.

The term “mismatch region” refers to a double-stranded region in a nucleic acid molecule or modified nucleic acid molecule, wherein the bases or modified bases do not form Watson-Crick base-pairing. The mismatch region is flanked by two base-paired regions. The double-stranded region can be non-hydrogen bonded, or hydrogen bonded to form Hoogsteen basepairs, etc, or both.

The term “mutually complementary region” refers to a region in a nucleic acid molecule or modified nucleic acid molecule that is Watson-Crick base paired.

The term “non-complementary region” refers to a region in a nucleic acid molecule or modified nucleic acid molecule that is not Watson-Crick base paired. For example, the non-complementary region can be designed to have bulged nucleotides, a single-stranded loop, overhang nucleotides at the 5′ or 3′ ends, or mismatch regions.

The term “stem region” refers to a region in a nucleic acid molecule or modified nucleic acid molecule that has at least two Watson-Crick basepairs. For example, the stem region can be designed to have more than one mutually complementary region linked by non-complementary regions, or form a continuous mutually complementary region.

The term “stem-loop structure” refers to a nucleic acid molecule or modified nucleic acid molecule with a single-stranded loop sequence flanked by a pair of 5′ and 3′ oligonucleotide or modified oligonucleotide arms. The 5′ and 3′ arms form the stem region.

The term “three-arm junction structure” refers to a strand of nucleic acid or modified nucleic acid that has a conformation of a stem region, a first stem-loop region, and a second stem-loop region linked together via arms of the stem regions. The first stem-loop region is 5′ to the second stem-loop region. The three regions can be connected via a non-complementary region, a phosphodiester linkage, or a modified phosphodiester linkage, or a combination thereof.

Unless otherwise defined, all technical and scientific terms used herein have the same meaning as commonly understood by one of ordinary skill in the art. In case of conflict, the present specification, including definitions, will control. Further, unless otherwise required by context, singular terms shall include pluralities and plural terms shall include the singular. Generally, nomenclatures used in connection with, and techniques of, cell and tissue culture, molecular biology, immunology, microbiology, genetics, developmental biology, cell biology described herein are those well-known and commonly used in the art.

Throughout this specification and the embodiments, the word “comprise,” or variations such as “comprises” or “comprising” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

### 2. Methods of the Invention

Two culture parameters that affect total RNA and protein production in a population of cells include: (i) cell specific production rate, and (ii) the growth characteristics of the population of cells used for RNA and/or protein production. The methods and compositions of the present invention optimize one or both of these parameters. A third variable that may influence protein production in a population of cells is the rate of proliferation of the cells. For example, cells with an increased rate of proliferation may attain a certain biomass of protein producing cells in a shorter period of time compared to cells with a decreased proliferation rate. Thus, the amount of protein produced in a given period of time is maximized. In some cases, cells may decrease cellular proliferation to shift energy output to protein production. Thus, in another embodiment, the invention provides a method for isolating a cell with a decreased rate of proliferation, wherein the cell also expresses increased levels of a protein encoding RNA of interest.

The methods of the present invention are based upon the ability of fluorogenic signaling probes and reagents that may be used as proliferation markers to produce a detectable signal in viable cells, without the need for fixing or lysing the cells. Fluorogenic signaling probes produce a detectable signal upon hybridization to target RNA sequences in living cells, and may relate to the amount of the corresponding protein that a cell produces when the RNA is a protein encoding RNA. The signal produced by a signaling probe and proliferation marker used in the invention should be detectably higher or different than the average produced in the tested population of cells (e.g., background fluorescence). Thus, it is not necessary that the average cells produce no fluorescence at all. In one embodiment, the invention provides a method for isolating cells or generating cell lines with increased production of an RNA or protein of interest. For example, the methods of the invention may be used to isolate a cell or to generate a cell line with increased production of an RNA or protein of interest when compared to production of an RNA or protein of interest in cells of the population that is tested. As used herein, a control cell is a cell that is identical to an isolated cell of the invention, but has not been selected for increased or decreased RNA or protein production, or has not been selected for an altered rate of cell proliferation, with any of the methods of the invention described herein.

### Generating Cell Lines Optimized for Increased RNA or Protein Production

The use of signaling probes and reagents that may be used as proliferation markers as described herein allow for the identification of cells optimized for increased production of an RNA of interest (e.g., an RNA that encodes a protein). Unlike reagents that require fixing or lysing of cells to analyze intracellular components, fluorescence from signaling probes and proliferation markers can be used to analyze intracellular RNA and protein levels in live cells. This characteristic allows one to isolate and propagate cells with increased production of an RNA of interest. In one embodiment, following the introduction of genetic sequence(s) encoding a protein(s) of interest into cells (e.g., transfection) with a DNA construct comprising a gene that encodes an RNA of interest, one introduces into cells fluorogenic signaling probes that recognize an RNA of interest. This step can be performed following optional selection using a selection marker, e.g., drug selection provided that the transfected DNA construct also encodes drug resistance. The cells that transcribe the gene will fluoresce.

In another embodiment, cells are contacted with a proliferation marker, such as CFSE. Following staining with CFSE, cell division may be monitored over time or cell division may be allowed to occur over a period of time prior to quantification of the signal of the proliferation marker. The cells may be allowed to proliferate from less than 1 hour to 1 day, from 1 to 5 days, from 3 to 10 days, from 5 to 15 days, from 1 week to 2 weeks, from 1.5 weeks to 4 weeks or up to 20 weeks prior to quantification of the signal from the proliferation marker. In one embodiment, cells with an increased rate of cell proliferation are isolated. Isolation of these cells may be based, for example, on a decreased fluorescent signal of the proliferation marker. Cells with varying rates of proliferation may be isolated based on differing levels of signal from the proliferation marker. In another embodiment, cells with a decreased rate of cell proliferation are isolated, wherein the cells express increased levels of an RNA or protein of interest. Cells may be exposed to the proliferation marker and the signaling probe simultaneously or at different times. If at different times, the cells may be exposed to the proliferation marker before or after exposure to the signaling probe. Cells may be exposed to the proliferation marker and the signaling probes at different times but analyzed at the same time, for instance cell may be exposed to the proliferation marker at one time and to the signaling probe at a second time following a period of time corresponding to the length of time required for several cell divisions based on average cell doubling times.

Cells that fluoresce at varying levels from CFSE staining or signal probe hybridization can be isolated using any known techniques for detecting fluorescence. For example, cells that fluoresce from CFSE staining or signal probe hybridization can be isolated by flow cytometric cell sorting technology. Isolated cells may then be used to produce cell lines that express high levels of the RNA of interest and that also have an increased rate of proliferation or a decreased rate of proliferation.

The methods and compositions of the present invention may also be used to isolate cells with increased production of more than one RNA of interest, even without the need to maintain the cells in the presence of selective drugs or agents. Cells can be transfected or otherwise introduced with two or more DNA or RNA constructs. The cells may be transfected with the two or more DNA or RNA constructs simultaneously or sequentially. The signaling probe for the first RNA of interest may produce the same or a different signal from the signaling probes for the other RNAs of interest. For example, they may have the same or different fluorophores. Cells or cell lines expressing more than two RNAs may be provided by repeating the steps simultaneously or sequentially. The DNA or RNA constructs optionally comprise one or more drug or selective agent markers. Following transfection, and optionally drug-selection, a signaling probe that is directed to each RNA of interest is introduced into the cells. In one embodiment, the cells are then sorted by flow cytometric cell sorting technology, thus isolating cells that express any combination of the two or more RNAs or proteins of interest.

In certain embodiments, multiple rounds of the methods described herein may be used to obtain cells with increased expression of two or more RNAs or proteins of interest. For example, cells may be transfected with one or more RNA or DNA constructs that encode an RNA or protein of interest and isolated according to the methods described herein. The isolated cells may then be subjected to further rounds of transfection with one or more other RNA or DNA constructs that encode an RNA or protein of interest and isolated once again. This method is useful, for example, for generating cells with increased expression of a complex of proteins, RNAs or proteins in the same or related biological pathway, RNAs or proteins that act upstream or downstream of each other, RNAs or proteins that have a modulating, activating or repressing function to each other, RNAs or proteins that are dependent on each other for function or activity, or RNAs or proteins that share homology (e.g., sequence, structural, or functional homology). For example, this method may be used to generate a cell line with increased expression of the heavy and light chains of an immunoglobulin protein (e.g., IgA, IgD, IgE, IgG, and IgM) or antigen-binding fragments thereof. The immunoglobulin proteins may be fully human, humanized, or chimeric immunoglobulin proteins.

Methods for Isolating Cells with Altered Rates of Cell Proliferation

In one embodiment, the invention provides a method for isolating cells or generating cell lines from a population of cells with an increased rate of cell proliferation when compared to the average growth of cells in the population. In another embodiment, the invention provides a method for isolating cells or generating cell lines from a population of cells with a decreased rate of cell proliferation when compared to the average growth of cells in the population. The cells may optionally also express increased levels of an RNA or protein of interest. The cell proliferation rate of cells isolated from a starting population may be increased or decreased at least 1.3-fold when compared to the average proliferation rate of cells in the starting population. In other embodiments, the cell proliferation rate of cells derived from cells isolated from starting populations is increased or decreased at least 1.5-fold, at least 2.0-fold, at least 2.5-fold, at least 3.0 fold, at least 5-fold, or at least 10-fold when compared to the average proliferation rate of cells in the starting population. In one embodiment, the rate of cell proliferation may be altered (e.g., increased or decreased) by optimization of media formulation (e.g., optimization of nutrient concentration, such as sugars, salts, amino acids, vitamins, etc.). In another embodiment, the rate of cell proliferation is altered by genetic or metabolic engineering. For example, the rate of cell proliferation may be altered by expressing, overexpressing, or altering the expression of genes or proteins that affect the rate of cell proliferation. See, for example, Mazur et al., Biotechnol Prog. 1998; 14:705-713, incorporated herein by reference in its entirety. In one embodiment, cell proliferation rate is altered by expressing, overexpressing, or altering the expression of genes or proteins responsible for the cell cycle, cell division, or DNA replication, such as, for example, genes that encode: cyclins, cyclin-dependent kinases, cell cycle dependent phosphatases, inhibitors of cyclin-dependent kinases, cell cycle transcription factors, DNA polymerases, histones and proteins that participate in the initiation of DNA replication. The specific genes or proteins that are genetically or metabolically engineered will depend on the organism being used in the invention and can be determined by a person of skill in the art. In one embodiment, cell proliferation rate is altered by expressing one or more MYC genes, such as c-MYC. See, for example, Ifandi et al., Biotechnol Prog. 2005; 21:671-677, incorporated herein by reference in its entirety.

In another embodiment, the invention provides a method for increasing the cell density in a cell culture when compared to the average cell density of a cell culture of cells from the starting cell culture population. The cells may optionally also express increased levels of an RNA or protein of interest. The cell density of a cell culture may be increased at least 1.2-fold when compared to the average cell density of cells from the starting cell culture population. In other embodiments, the cell density is increased at least 1.5-fold, at least 2.0-fold, at least 2.5-fold, at least 3.0 fold, at least 5-fold, or at least 10-fold when compared to the average cell density of a cell culture of cells from the starting cell population. In one embodiment, the cell culture density is increased by optimization of media formulation (e.g., optimization of nutrient concentration, such as sugars, salts, amino acids, vitamins, etc.). In another embodiment, the cell culture density is increased by genetic or metabolic engineering. For example, of apoptosis suppressed in cells by expressing Bcl-2, Bcl-X_{L}, or p21^{CIP1}. The suppression of apoptosis may increase the density of a culture of cells as well as increase protein production. See, for example, Itoh et al., Biotechnology and Bioengineering 2004; 48:118-122; Chiang et al., Biotechnology and Bioengineering 2005; 91:779-792; and, Jung et al., Biotechnology and Bioengineering 2002; 79:180-187, incorporated herein by reference in their entirety. In certain embodiments, one or more MYC genes are co-expressed with Bcl-2 to increase both the rate of cell proliferation and cell density. See, for example, Ifandi et al., Biotechnol. Prog. 2005; 21:671-677 and Bissonnette et al., Nature. 1992; 359:552-554, incorporated herein by reference in their entirety.

The rate of cell proliferation will vary according to the type of cell used in the methods of the invention. For example, a bacterial cell may divide to produce two viable daughter cells in 30 minutes or less, whereas a mammalian cell may divide once every 10-24 hours, or may take more than one day per cell division. For example, a eukaryotic cell may divide once every 12-30 hours. Doubling times for a cell can be determined by the skilled worker by monitoring the increase in the number of viable cells in a population over the proliferative phase of a cell's growth cycle.

An increase in cell proliferation will also depend on nutritional conditions. In certain embodiments, the proliferation rate of a cell is increased by optimization of media formulation (e.g., optimization of nutrient concentration, such as sugars, salts, amino acids, vitamins, etc.). See, for example, Chu and Robinson, Curr Opin Biotechnol. 2001 April; 12(2):180-7; Chun et al., Biotechnol Prog 2003 January-February; 19(1):52-7; Dempsey et al., Biotechnol Prog 2003 January-February; 19(1):175-8; and Sauer et al., Biotechnol Bioeng. 2000 Mar. 5; 67(5):585-97, incorporated herein by reference in their entirety.

Environmental conditions may also be optimized for increased recombinant protein yield. For example, subjecting mammalian cells to sub-physiological temperatures may lead to an increase in recombinant protein yield. See, for example, Al-Fageeh et al., Biotechnology and Bioengineering. 2006 93:829-835 and Baik et al., Biotechnology and Bioengineering. 2006 93:361-371, incorporated herein in their entirety.

Cells may be quantitated using standard methods and instrumentation. For example, a portion of the cells can be plated on solid growth media to measure the number of colony forming cell units in the population. Alternatively, instruments such as a spectrophotometer or haemocytometer may be used. Automated techniques and instruments for measuring cell density such as the Guava ViaCount assay and the Beckman Coulter Vi-CELL automated cell viability analyzer may also be used.

In any of the methods of the invention for isolating cells, the cells may be cultured to produce a cell culture or to generate cell lines.

Any of the methods described herein for isolating cells or generating cell lines with an increased rate of cell proliferation may also comprise the step of monitoring cells for an apoptotic or pre-apoptotic marker. Apoptotic and pre-apoptotic markers include, for example, DNA cleavage, nuclear fragmentation, chromosome condensation, necrosis, blebbing of the cell membrane, cleavage of poly(ADP-ribose) polymerase, caspase 3 activation, or expression of other genes involved in apoptosis. Apoptotic or pre-apoptotic markers also include permeability to propidium iodide or 7-AAD. In one embodiment, the methods described herein further comprise the step of contacting a cell with an increased rate of proliferation or increased RNA or protein production with a reagent for monitoring an apoptotic or pre-apoptotic marker. For instance, a cell that exhibits increased fluorescence of a fluorogenic signaling probe and altered fluorescence of a reagent for monitoring the rate of cell proliferation may be contacted with a reagent for monitoring an apoptotic or pre-apoptotic marker. Agents for monitoring an apoptotic or pre-apoptotic marker are well known in the art. Examples of fluorescent reagents for monitoring an apoptotic or pre-apoptotic marker include fluorescently labeled Annexin-V and propidium iodide.

Methods for Producing a Cell Culture with Increased Density

In one embodiment, the invention provides a method for producing a cell culture with greater propensity for_increased cell density. For example, the number of viable cells capable of protein production, or biomass, is increased (e.g., the cell culture density is increased) so that the total amount of protein produced by the biomass is increased. Cell culture density may be increased by one to ten-fold (e.g., by 1.5-fold, 2-fold, 3-fold, 5-fold, or 10-fold), by ten to 100-fold (e.g., by 15-fold, 25-fold, 50-fold, or 100-fold), by 100 to 1000-fold (e.g., by 150-fold, 250-fold, 500-fold or 1000-fold), or by greater than 1000-fold. For example, final cell culture density may range from approximately 1×10⁴ cells/ml to 1×10⁵ cells/ml of culture, from 1×10⁵ cells/ml to 1×10⁶ cells/ml of culture, from 1×10⁶ cells/ml to 1×10⁷ cells/ml of culture, from 1×10⁷ cells/ml to 1×10⁸ cells/ml of culture, or even greater that 1×10⁸ cells/ml of culture. The increase in cell culture density will depend on nutritional and environmental conditions, and will vary according to the types of cells used in the invention. Cells isolated according to various levels of labeling with one or more proliferation markers may be cultured and resulting populations may be tested to identify those with a greater propensity to achieve higher cell densities.

In certain embodiments, a biomass of protein producing cells is increased by optimization of media formulation (e.g., optimization of nutrient concentration, such as sugars, salts, amino acids, vitamins, etc.). See, for example, Chu and Robinson, Curr Opin Biotechnol. 2001 April; 12(2):180-7; Chun et al., Biotechnol Prog 2003 January-February; 19(1):52-7; Dempsey et al., Biotechnol Prog 2003 January-February;19(1):175-8; and Sauer et al., Biotechnol Bioeng. 2000 Mar. 5; 67(5):585-97, incorporated herein by reference in their entirety. In another embodiment, a biomass of protein producing cells is increased by preventing cell death or apoptosis in a population of protein producing cells. In other embodiments, the invention provides methods for producing a cell culture with increased cell density, wherein the cells in the cell culture express increased levels of a RNA of interest. For instance, a method for producing a high concentration of viable and productive cells that also proliferates rapidly is provided.

In another embodiment, the invention provides a method of altering the cell culture density of a population of cells by genetic or metabolic engineering. For example, cell density may be increased by inhibiting apoptotic cell death. In one embodiment, anti-apoptotic survival proteins are expressed, such as bcl-2 or bcl-xL. In other embodiments, caspase inhibition or expression of the molecular chaperone HSP70 is used to increase cell density. In other embodiments, metabolic engineering approaches may be used. Metabolic engineering may be used to increase cell density by inhibiting the accumulation of toxic by-products of metabolism, such as lactate and ammonia, or by engineered improvement of metabolic pathways. For example, pyruvate carboxylase expression may increase flux of glucose into the tricarboxylic acid cycle.

Any of the methods described herein for producing a cell culture with increased cell density may also comprise the step of monitoring cells for apoptotic cell death. In one embodiment, the methods described herein further comprise the step of contacting a cell in a cell culture with a reagent for monitoring an apoptotic or pre-apoptotic marker. Agents for monitoring apoptotic or pre-apoptotic markers are well known in the art. Examples of fluorescent reagents for monitoring apoptosis include fluorescently labeled Annexin-V and propidium iodide. In certain embodiments, apoptotic cells or cells with a propensity for apoptosis are negatively selected.

Methods for Isolating a Cell with a Biphasic Growth Profile

The invention provides a method for isolating a cell with a biphasic growth profile. A biphasic growth profile can be characterized by rapid proliferation in a first portion of the growth profile. This rapid proliferation allows for the accumulation of a population of protein producing cells in a short period of time. The rapid period of growth is then followed by a shift from rapid to slow proliferation or no proliferation. The period of decreased or lower proliferation may be characterized by increased protein production. Thus, a cell isolated according to the methods of the present invention may have an increased proliferation rate in the first portion of its growth profile. This portion of the growth profile may also be characterized by an increased or decreased protein production rate. In the second portion of the growth profile, the cell or cells have a decreased or lower proliferation rate, wherein the cell expresses increased levels of an RNA of interest as compared to expression levels during the first portion of the growth profile. In certain embodiments, the cells are also monitored for cell death and apoptosis to select for a population of cells that has minimal presence of apoptotic or preapoptotic markers.

### Methods of Detecting Fluorescence

Fluorescence cell sorter or related technology can be used with fluorogenic probes or proliferation markers to identify and/or separate cells exhibiting a certain level or levels of fluorescence at one or more wavelengths. For instance, fluorescence of signaling probes and proliferation markers may be detected and/or quantitated by fluorescence microscopy, fluorocytometry, flow cytometric cell sorting technology, or by a fluorescent plate reader. Flow cytometric cell sorting technology currently allows sorting at up to 70,000 cells per second. 5,000,000 cells can be sorted in less than 2 minutes.

The methods described herein may also be used simultaneously with assays that utilize a fluorescent reporter for the detection of intracellular events, states or compositions (e.g., apoptosis, necrosis, Ca2+/Ion flux, pH flux, cell adhesion, cell division and growth, or DNA content). Examples of fluorescent assays that detect intracellular events include, for example, fluorescent staining (e.g., of nucleic acids, proteins and/or membranes), and assays used to detect interactions between proteins or between proteins and nucleic acids. Reagents which may be fluorescently labeled for use in these assays include but are not limited to proteins (labeled with fluorescent molecules or autofluorecent proteins); fluorescent metabolic indicators (e.g., C12 resazurin); fluorescent substrates or by-products; fluorescently-labeled lectins; fluorescent chemicals; caged fluorescent compounds; fluorescent nucleic acid dyes; and fluorescent polymers, lipids, amino acid residues and nucleotide/side analogues.

### Cells

The methods of the invention may be used with any cell that is suitable for use with the signaling probes and proliferation markers described herein. In one embodiment, the cells are selected from the group consisting of mammalian cells, bacterial cells, plant, microbial, algal and fungal cells. In some embodiments, the cells are mammalian cells, such human, mouse, rat, goat, horse, rabbit, hamster or cow cells. For instance, the cells may be from any established cell line, including but not limited to HeLa, NS0, SP2/0, HEK 293T, Vero, Caco, Caco-2, MDCK, COS-1, COS-7, K562, Jurkat, CHO-K1, DG44, CHOK1SV, CHO-S, Huvec, CV-1, HuH-7, NIH3T3, HEK293, 293, A549, HepG2, IMR-90, MCF-7, U-20S, Per.C6, SF9, SF21 or Chinese Hamster Ovary (CHO) cells. In certain embodiments, the cells are fungal cells, such as cells selected from the group consisting of: Chrysosporium cells, Aspergillus cells, Trichoderma cells, Dictyostelium cells, Candida cells, Saccharomyces cells, Schizosaccharomyces cells, and Penicillium cells. In certain other embodiments, the cells are bacterial cells, such as E. coli, B. subtilis, or BL21 cells.

### Recombinant DNA Constructs

In one embodiment, the methods and compositions of the invention are used to isolate cells with increased production of an RNA of interest (e.g., an RNA that encodes a protein of interest). An RNA of interest may be expressed from a gene on a DNA construct. For example, a DNA construct that is transcribed into an RNA of interest is introduced into cells. The DNA construct may be integrated at different locations in the genome of the cell or may remain in the cytoplasm of the cell. Integration at one or more specific loci may also be accomplished. Then, the transfected cells are exposed to the signaling probe and/or the proliferation marker. The signaling probe and proliferation marker may be exposed to cells at the same time, immediately after one another, at entirely different times, and in any order. After the cells are exposed to the signaling probe and/or proliferation marker, a detectable signal is generated and the cells of interest are isolated. Cells can be isolated and cultured by any method in the art, e.g., cells can be isolated and plated individually or in batch. Cell lines can be generated by growing the isolated cells.

Any of the methods of the invention may be carried out using a selection marker. Although drug selection (or selection using any other suitable selection marker) is not a required step, it may be used to enrich a cell population for cells that are stably transfected with a DNA construct that encodes the protein of interest, provided that the transfected constructs are designed to confer drug resistance. If selection using signaling probes is performed too soon following transfection, some positive cells may only be transiently and not stably transfected. However, this can be minimized given sufficient cell passage allowing for dilution or loss of transfected plasmid from non-stably transfected cells or given multiple rounds of selection according to the methods described herein.

### Exemplary RNAs and Proteins of Interest

A DNA construct that is transfected into a cell of the invention may comprise a sequence that is transcribed into an RNA encoding a protein of interest that has one or more of the following different roles: messenger RNAs that encode proteins, fusion proteins, peptides fused to proteins, export signals, import signals, intracellular localization signals or other signals, which may be fused to proteins or peptides. Any protein may be produced according to the methods described herein. Examples of proteins that may be produced according the methods of the invention include, without limitation, peptide hormones (e.g., insulin), glycoprotein hormones (e.g., erythropoietin), antibiotics, cytokines, enzymes, vaccines (e.g., HIV vaccine, HPV vaccine, HBV vaccine), anticancer therapeutics (e.g., Muc1), and therapeutic antibodies. In a particular embodiment the RNA encodes an immunoglobulin protein or an antigen-binding fragment thereof, such as an immunoglobulin heavy chain, an immunoglobulin light chain, a single chain Fv, a fragment of an antibody, such as Fab, Fab′, or (Fab′)₂, or an antigen binding fragment of an immunoglobulin. In a specific embodiment, the RNA encodes erythropoietin. In another specific embodiment, the RNA encodes one or more immunoglobulin proteins, or fragments thereof, that bind to: the epidermal growth factor receptor (EGFR), HER1, or c-ErbB-1, such as Erbitux® (cetuximab). An RNA that is produced by the methods and compositions of the invention may also have one or more of the following roles: antisense RNA, siRNA, structural RNAs, cellular RNAs including but not limited to such as ribosomal RNAs, tRNAs, hnRNA, snRNA; random RNAs, RNAs corresponding to cDNAs or ESTs; RNAs from diverse species, RNAs corresponding to oligonucleotides, RNAs corresponding to whole cell, tissue, or organism cDNA preparations; RNAs that have some binding activity to other nucleic acids, proteins, other cell components or drug molecules; RNAs that may be incorporated into various macromolecular complexes; RNAs that may affect some cellular function; or RNAs that do not have the aforementioned function or activity but which may be expressed by cells nevertheless; RNAs corresponding to viral or foreign RNAs, linker RNA, or sequence that links one or more RNAs; or, RNAs that serve as tags or a combination or recombination of unmodified mutagenized, randomized, or shuffled sequences of any one or more of the above.

A DNA construct of the invention may comprise DNA that encodes an RNA of interest that is operatively linked to a constitutive or conditional promoter, including but not limited to inducible, repressible, tissue-specific, heat-shock, developmental, cell lineage specific, or temporal promoters or a combination or recombination of unmodified or mutagenized, randomized, shuffled sequences of any one or more of the above.

### 3. Signaling Probes

Nucleic acid probes that recognize and report the presence of a specific nucleic acid sequence have been used to detect specific nucleic acids. See, for example, U.S. Pat. No. 5,925,517, incorporated herein by reference in its entirety. One type of probe is designed to have a hairpin or stem-loop shaped structure, with a central stretch of nucleotides complementary to the target sequence, and termini comprising short mutually complementary sequences. See, for example, Tyagi and Kramer, Nature Biotechnology, 14, 303-308 (1996), incorporated herein by reference in its entirety. One terminus of the probe is covalently bound to a fluorophore and the other to a quenching moiety. When in their native state with hybridized termini, the proximity of the fluorophore and the quencher is such that relatively little or essentially no fluorescence is produced. The probe undergoes a conformational change when hybridized to its target nucleic acid that results in the detectable change in the production of fluorescence from the fluorophore. Such probes have been used to visualize messenger RNA in living cells (Matsuo, 1998, Biochim. Biophys. Acta 1379:178-184). Similar probes have been used to isolate living cells based on the expression of RNA sequences. See, for example, U.S. Pat. No. 6,692,965, incorporated herein by reference in its entirety.

Signaling probes used in the present invention are designed to be complementary to either a portion of the RNA of interest or to a portion of its 5′ or 3′ untranslated region. The gene that encodes the RNA of interest may be tagged with a tag sequence and the signaling probe may be designed so that it recognizes the tag sequence. The tag sequence can either be in frame with the protein-coding portion of the message of the gene or out of frame with it, depending on whether one wishes to tag the protein produced. Tag sequences can be any nucleotide sequence that is complementary or partially complementary to the sequence of the signaling probe. Examples of protein tags include, without limitation, c-myc, hemagglutinin, and glutathione S-transferase.

### Interacting Pair

The signaling probe comprises one or more interacting pairs, and may have different interacting pairs. In one embodiment, the signaling probe is a fluorogenic probe. See, for example, U.S. Pat. No. 6,692,965 and International Publication WO 2005/079462, hereby incorporated by reference in their entirety. In one embodiment, the fluorogenic probe does not emit or emits a background level of fluorescence in its unhybridized state, but fluoresces upon or fluoresces above the background level upon binding to its target. Multiple fluorophores can be used to increase signal or provide fluorescence at different color ranges. Multiple quenchers can be used to decrease or eliminate signal in the absence of target sequence. Examples of quenchers include but are not limited to DABCYL, EDAC, Cesium, p-xylene-bis-pyridinium bromide, Thallium and Gold nanoparticles. Examples of fluorophores include but are not limited to sulforhodamine 101, acridine, 5-(2′-aminoethyl) aminoaphthaline-1-sulfonic acid (EDANS), Texas Red, Eosine, and Bodipy and Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750, Allophycocyanin, Aminocoumarin, Bodipy-FL, Cy2, Cy3, Cy3.5, Cy5, Cy5.5, carboxyfluorescein (FAM), Cascade Blue, APC-Cy5, APC-Cy5.5, APC-Cy7, Coumarin, ECD (Red613), Fluorescein (FITC), Hexachlorfluoroscein (HEX), Hydroxycoumarin, Lissamine Rhodamine B, Lucifer yellow, Methoxycoumarin, Oregon Green 488, Oregon Green 514, Pacific Blue, PE-Cy7 conjugates, PerC, PerCP-Cy5.5, R-Phycoerythrin (PE), Rhodamine, Rhodamine Green, Rodamine Red-X, Tetratchlorofluoroscein (TET), TRITC, Tetramethylrhodamine, Texas Red-X, TRITC, XRITC, and Quantum dots. See, for example, Tyagi et al. Nature Biotechnology 16:49-53, (1998) and Dubertret et al., Nature Biotechnology, 19:365-370 (2001), incorporated herein by reference in their entirety.

The invention also provides signaling probes that are wavelength-shifting. In one embodiment, one terminus of the probe has at least a harvester fluorophore and an emitter fluorophore, an adjacent terminus of the probe has at least a quencher moiety. See, for example, Tyagi et al., Nature Biotechnology, 18, 1191-1196 (2000), incorporated herein by reference in its entirety. In one embodiment, the harvester fluorophore and the emitter fluorophore are at the same terminus, wherein the emitter fluorophore is at the distal end, and a quencher moiety is at an opposite terminus to the harvester fluorophore. The emitter fluorophore may be separated from the harvester fluorophore by a spacer arm of a few nucleotides. The harvester fluorophore absorbs strongly in the wavelength range of the monochromatic light source. In the absence of target sequence, both fluorophores are quenched. In the presence of targets, the probe fluoresces in the emission range of the emitter fluorophore. The shift in emission spectrum is due to the transfer of absorbed energy from the harvester fluorophore to the emitter fluorophore by fluorescence resonance energy transfer. These types of signaling probes may provide a stronger signal than signaling probes containing a fluorophore that cannot efficiently absorb energy from the monochromatic light sources. In one embodiment, the harvester fluorophore is fluorescein and the emitter fluorophore is 6-carboxyrhodamine 6G, tetramethylrhodamine or Texas red.

In another embodiment, one terminus of the probe has at least a fluorophore F1, and another adjacent terminus has at least another fluorophore F2. The two fluorophores are chosen so that fluorescence resonance energy transfer (FRET) will occur when they are in close proximity. When the probe is not bound to its target sequence, upon excitation at the absorption band of F1, the fluorescence of F1 is quenched by F2, and the fluorescence of F2 is observed. When the probe is bound to its target sequence, FRET is reduced or eliminated and the fluorescence of F1 will rise while that of F2 will diminish or disappear. This difference in fluorescence intensities can be monitored and a ratio between the fluorescence of F1 and F2 can be calculated. As residual fluorescence is sometimes observed in fluorophore-quencher systems, this system may be more advantageous in the quantitative detection of target sequence. See, Zhang et al., Angrew. Chem. Int. Ed., 40, 2, pp. 402-405 (2001), incorporated herein by reference in its entirety. Examples of FRET donor-acceptor pairs include but are not limited to the coumarin group and 6-carboxyfluorescein group, respectively.

In one embodiment, the signaling probe comprises a luminescent label and adduct pair. The interaction of the adduct with the luminescent label diminishes signal produced from the label. See Becker and Nelson, U.S. Pat. No. 5,731,148, incorporated herein by reference in its entirety.

In another embodiment, the signaling probe comprises at least a dye dimer. When the probe is bound to the target sequence, the signal from the dyes are different from the signal of the dye in dimer conformation.

### Conformation of Signaling Probes or Other Probes

### Double-stranded Structure

The present invention provides signaling probes or other probes comprising at least two separate strands of nucleic acid that are designed to anneal to each other or form at least a mutually complementary region. At least one terminus of one strand is adjacent to a terminus of the other strand. The nucleic acid may be DNA, RNA or modified DNA or RNA. The two strands may be identical strands that form a self-dimer. The strands may also not be identical in sequence.

The two separate strands may be designed to be fully complementary or comprise complementary regions and non-complementary regions. In one embodiment, the two separate strands are designed to be fully complementary to each other. In one embodiment, the two strands form a mutually complementary region of 4 to 9, 5 to 6, 2 to 10, 10 to 40, or 40 to 400 continuous basepairs at each end. The strands may contain 5-7,8-10, 11-15, 16-22, more than 30, 3-10, 11-80, 81-200, or more than 200 nucleotides or modified nucleotides. The two strands may have the same or a different number of nucleotides. For example, one strand may be longer than the other. In one embodiment, the 5′ end of one strand is offset from the other strand, or the 3′ end of that strand is offset from the other strand, or both, wherein the offset is up to 10, up to 20, or up to 30 nucleotides or modified nucleotides.

The region that hybridizes to the target sequence may be in the complementary regions, non-complementary regions of one or both strands or a combination thereof. More than one target nucleic acid sequence may be targeted by the same signaling probe. The one or more targets may be on the same or different sequences, and they may be exactly complementary to the portion of the probe designed to bind target or at least complementary enough. In one embodiment, the two strands form a mutually complementary region at each end and the target complement sequence resides in the regions other than the mutually complementary regions at the ends.

In one embodiment, the signaling probe with at least two separate strands is a fluorogenic probe. In one embodiment, one strand has at least a quencher moiety on one terminus, and a fluorophore on an adjacent terminus of the other strand. In one embodiment, each of the 5′ and 3′ terminus of one strand has the same or a different fluorophore, and each of the 5′ and 3′ terminus of the other strand has the same or a different quencher moiety. In one embodiment, the 5′ terminus of one strand has a fluorophore and the 3′ terminus has a quencher moiety, and the 3′ terminus of the other strand has the same or a different quencher moiety and the 5′ terminus has the same or a different fluorophore.

### Stem-loop Structure

In another embodiment, the signaling probe is a strand of nucleic acid or modified nucleic acid that comprises at least a mutually complementary region and at least a non-complementary region. In one embodiment, the probe forms a stem-loop structure. The stem region can be mutually complementary, or comprise mutually complementary regions and non-complementary regions. For example, the stem region can have bulged nucleotides that are not base-paired. The stem region can also contain overhang nucleotides at the 5′ or 3′ ends that are not base-paired.

When the stem region is fully complementary, the stem region can include 3-4,5-6, 7-8, 9-10, 2-6, 7-10, or 11-30 base-pairs. The loop region can contain 10-16, 17-26, 27-36, 37-45, 3-10, 11-25, or 25-60 nucleotides. In one embodiment, the stem region forms 4-10, 4, or 5 continuous basepairs.

In one embodiment, the stem-loop structure comprises at least an interactive pair comprising two chemical groups, and one chemical group is at each terminus of the strand. In one embodiment, the signaling probe has at least a fluorophore and a quencher moiety at each terminus of the strand.

In one embodiment, the stem region comprises two mutually complementary regions connected via a non-complementary region, the mutually complementary region adjacent to the interactive pair forms 5 to 9 basepairs, and the mutually complementary region adjacent to the loop region forms 4 to 5 basepairs. In one embodiment, the non-complementary region is a single-stranded loop region, a mismatch region or both. In another embodiment, the stem region comprises three mutually complementary regions connected via two non-complementary regions, the first mutually complementary region adjacent to the interactive pair forms 4 to 5 basepairs, the second mutually complementary region forms 2 to 3 basepairs, and the third mutually complementary region adjacent to the loop region forms 2 to 3 basepairs.

In the stem-loop structure, the region that is complementary to the target sequence may be in one or more stem regions or loop regions, or both. The region in the stem that hybridizes to the target may be in the mutually complementary regions, non-complementary regions or both. In one embodiment, the target complement sequence is in the single-stranded loop region. In one embodiment, the regions other than the stem region adjacent to the interactive pair is the target complement sequence. More than one target nucleic acid sequence may be targeted by the same probe. The one or more targets may be on the same or different sequences, and they may be exactly complementary to the portion of the probe designed to bind target or at least complementary enough.

The increase in stem length may increase the stability of the signaling probes in their closed conformation, and thus, may increase the signal to noise ratio of detectable signal. Exposure of these signaling probes to cells can be carried out at slightly elevated temperatures which are still safe for the cell followed by a return to normal temperatures. At the higher temperatures, the signaling probes would open and bind to their target if present. Once cooled, the signaling probes not bound to target would revert to their closed states, which is assisted by the increased stability of the stem. Similarly, other forces may be used to achieve the same outcome, for instance DMSO which is thought to relax base-pairing.

### Chemical Modification of Signaling Probes

The present invention also provides signaling probes or other probes which are chemically modified. One or more of the sugar-phosphodiester type backbone, 2′OH, base can be modified. The substitution of the phosphodiester linkage includes but is not limited to —OP(OH)(O)O—, —OP(O⁻M⁺)(O)O—, —OP(SH)(O)O—, —OP(S⁻M⁺)(O)O—, —NHP(O)₂O—, —OC(O)₂O—, —OCH₂C(O)₂ NH—, —OCH₂C(O)₂O—, —OP(CH₃)(O)O—, —OP(CH₂C₆H₅)(O)O—, —P(S)(O)O— and —OC(O)₂NH—. M⁺ is an inorganic or organic cation. The backbone can also be peptide nucleic acid, where the deoxyribose phosphate backbone is replaced by a pseudo peptide backbone. Peptide nucleic acid is described by Hyrup and Nielsen, Bioorganic & Medicinal Chemistry 4:5-23, 1996, and Hydig-Hielsen and Godskesen, WO 95/32305, each of which is hereby incorporated by reference herein in their entirety.

The 2′ position of the sugar includes but is not limited to H, OH, C₁-C₄ alkoxy, OCH₂—CH═CH₂, OCH₂—CH═CH—CH₃, OCH₂—CH═CH—(CH₂)ₙCH3 (n=0, 1 . . . 30), halogen (F, Cl, Br, I), C₁-C₆ alkyl and OCH₃. C₁-C₄ alkoxy and C₁-C₆ alkyl may be or may include groups which are straight-chain, branched, or cyclic.

The bases of the nucleotide can be any one of adenine, guanine, cytosine, thymine, uracil, inosine, or the forgoing with modifications. Modified bases include but are not limited to N4-methyl deoxyguanosine, deaza or aza purines and pyrimidines. Ring nitrogens such as the N1 of adenine, N7 of guanine, N3 of cytosine can be alkylated. The pyrimidine bases can be substituted at position 5 or 6, and the purine bases can be substituted at position 2, 6 or 8. See, for example, Cook, WO 93/13121; Sanger, Principles of Nucleic Acid Structure, Springer-Verlag, New York (1984), incorporated herein by reference in their entirety.

Derivatives of the conventional nucleotide are well known in the art and include, for example, molecules having a different type of sugar. The O4′ position of the sugar can be substituted with S or CH₂ For example, a nucleotide base recognition sequence can have cyclobutyl moieties connected by linking moieties, where the cyclobutyl moieties have hetereocyclic bases attached thereto. See, e.g., Cook et al., International Publication WO 94/19023 (hereby incorporated by reference herein in its entirety).

Other chemical modifications of probes useful in facilitating the delivery of the probes into cells include, but are not limited to, cholesterol, transduction peptides (e.g., TAT, penetratin, etc.).

### 4. Proliferation Markers

In certain embodiments, fluorescent markers of cell division are used in the methods of the invention. Fluorescent markers that label cells are useful for monitoring cell division because alterations in the fluorescence of the labeled cell indicate that a cell has divided. The fluorescence can be monitored over time to establish a rate of cell proliferation. An increase in cell proliferation will correlate with either an increase or decrease of the fluorescent marker that is used to label the cell. In one embodiment, a decrease in fluorescence of the fluorescent marker of cell division correlates with an increase in cell proliferation. In another embodiment, an increase in fluorescence of the fluorescent marker of cell division correlates with an increase in cell proliferation.

In a particular embodiment, cells are labeled with carboxyfluorescein diacetate succinimidyl ester (CFSE), a fluorescent dye that spontaneously and irreversibly binds to cellular proteins by reaction with lysine side chains and other available amine groups. CFSE dye is loaded into cells in vitro and fluorescence monitored over time; cell division is allowed over a period of time prior to analysis of the cell population for CSFE labeling. Upon division, CFSE segregates equally between daughter cells so that the intensity of fluorescence within a cell decreases twofold with each successive generation. This property of CFSE allows accurate tracking of the number of divisions that a given cell has undergone (Weston and Parish, J Immunol Methods. 1990 Oct. 4; 133(1):87-97; Lyons and Parish, J Immunol Methods. 1994 May 2; 171(1):131-7; Parish, Immunol Cell Biol. 1999 December; 77(6):499-508; Lyons, J Immunol Methods. 2000 Sep. 21; 243(1-2):147-54). The fluorescent intensity of a cell labeled with CFSE can be detected by any device that is suitable for monitoring fluorescent signals, such as for example, a flow cytometric cell sorter, a fluorocytometer, a fluorescence microscope, or a fluorescence plate reader.

Other fluorescent markers of cell division that may be used in the invention include, without limitation, CFSE derivatives, carboxylic acid diacetate succinimidyl ester dyes and their derivatives, such as the succinimidyl ester of Oregon Green 488 carboxylic acid diacetate (carboxy-DFFDA SE), 5-(and -6)-carboxyeosin diacetate succinimidyl ester (CEDA SE), PKH26, Hoechst CPAI, Cyquant GR and NF dyes, MTT, CTT, and SNARF-1 carboxylic acid, acetate succinimidyl ester.

### EXAMPLES

### Example 1

### Cells with Biphasic Growth Profiles that Produce Increased Levels of an RNA of Interest

Cells are transfected with a recombinant DNA plasmid (e.g., that encodes a single-chain Fv immunoglobulin fragment that binds to the epidermal growth factor receptor). Standard methods of transfecting cells are well known. Cell transfection can be accomplished through a variety of methods using commercially available reagents or kits (Qiagen, Promega, Invitrogen, Stratagene) and following the manufacturer's instructions. If necessary, the cells may be separated from each other by standard and well established methods such as by homogenization and further chemical treatment. Cells are then exposed to a selective antibiotic, of which resistance is conferred by the same (or a different) plasmid, to enrich for cells integrating the recombinant gene of interest into the cells' genomes. The cells are then stained with carboxyfluorescein diacetate succinimidyl ester (CSFE) and grown at low density for a defined period of time. The cells are then exposed to a fluorogenic probe that hybridizes to an RNA of interest (e.g., an RNA that encodes a single-chain Fv immunoglobulin fragment that binds to the epidermal growth factor receptor). The fluorogenic probe is selected so that fluorescence of the probe increases when it hybridizes to the RNA of interest. Cells are selected on the basis of their more rapid loss of CFSE fluorescence and their high degree of fluorogenic probe fluorescence (cells that are growing rapidly at low density while maintaining a high level of RNA expression of the gene(s) of interest). Isolated cells are allowed to grow to ample numbers.

The isolated cells are stained with CFSE again and grown at high density. Periodically over several days, flow cytometry is performed. Cells are isolated based on two criteria: 1) a decrease in the rate of loss of fluorescence of CFSE in the higher density cell cultures and 2) increased fluorescence of the fluorogenic probe. The cells are also stained with propidium iodide or Annexin-V to eliminate apoptotic cells. The highest density cell culture may depend on the maintenance of increased fluorescence of the fluorogenic probe and low levels of apoptosis. Flow cytometry is performed on the resulting cell culture to isolate a cell clone that has a biphasic growth profile, can produce high levels of an RNA of interest, and can grow to high density with low levels of apoptosis.

What is claimed is:

1. A method for isolating a cell with an increased rate of cell proliferation, comprising the steps of:
contacting a population of cells with a fluorescent reagent for monitoring the rate of cell proliferation; and
isolating the cell that exhibits a level of fluorescence of the fluorescent reagent that correlates with increased cell proliferation.

2. The method of claim 1, wherein detection of fluorescence is carried out using flow cytometric cell sorting technology.

3. A method for isolating a cell with an increased rate of cell proliferation, wherein the cell also expresses high levels of an RNA of interest, comprising the steps of:
contacting a population of cells with a fluorogenic probe that fluoresces upon hybridization to said RNA of interest;
contacting said population with a fluorescent reagent for monitoring the rate of cell proliferation, wherein the reagent fluoresces at a wavelength different than that of the fluorogenic probe; and
isolating the cell that exhibits increased fluorescence of the fluorogenic probe and a level of fluorescence of the fluorescent reagent that correlates with increased cell proliferation.

4. The method of claim 3, wherein detection of the fluorescence of the fluorogenic probe is assayed simultaneously with detection of the fluorescence of the fluorescent reagent.

5. The method of claim 3, wherein detection of fluorescence is carried out using flow cytometric cell sorting technology.

6. The method of claim 3, wherein said fluorescent reagent for monitoring the rate of cell proliferation is selected from the group consisting of: carboxyfluorescein diacetate succinimidyl ester, SNARF-1 carboxylic acid or acetate succinimidyl ester.

7. The method of claim 3, wherein said cell is a mammalian, bacterial, insect, plant, microbial, algal or fungal cell.

8. The method of claim 7, wherein said mammalian cell is selected from the group consisting of: a Chinese Hamster Ovary (CHO) cell, a NS0 cell, a HEK 293 cell, and a Per.C6 cell.

9. The method of claim 7, wherein said bacterial cell is a BL21 cell.

10. The method of claim 7, wherein said fungal cell is selected from the group consisting of: a Chrysosporium cell, an Aspergillus cell, a Trichoderma cell, a Dictyostelium cell, a Candida cell, a Saccharomyces cell, a Schizosaccharomyces cell and a Penicillium cell.

11. The method of claim 7, wherein said insect cell is a SF9 cell or a SF21 cell.

12. The method of claim 3, further comprising the step of contacting the cell that exhibits increased fluorescence of the fluorogenic probe and altered fluorescence of the fluorescent reagent with a reagent for monitoring an apoptotic or pre-apoptotic marker.

13. The method of claim 12, wherein said reagent is Annexin-V or propidium iodide.

14. The method of claim 3, wherein the RNA of interest is selected from the group consisting of: a messenger RNA that encodes a protein, an antisense RNA molecule, a structural RNA, a ribosomal RNA, an hnRNA, and an snRNA.

15. The method of claim 14, wherein said messenger RNA encodes an immunoglobulin heavy chain, an immunoglobulin light chain, a single chain Fv, an Fab, Fab′, or (Fab′)₂ antibody fragment or an antigen binding fragment of an immunoglobulin.

16. The method of claim 3, wherein the RNA of interest is an endogenous RNA.

17. The method of claim 3, wherein the RNA of interest is a heterologous RNA.

18. The method of claim 3, further comprising the step of culturing the isolated cell to produce a cell culture.

19. The method of claim 18, further comprising the step of measuring the density of the cell culture.

20. A method of producing a cell culture with increased cell density, wherein cells in the cell culture express high levels of an RNA of interest, comprising the steps of:
contacting a population of cells with a fluorogenic probe that fluoresces upon hybridization to said RNA of interest;
isolating a cell from the population that exhibits increased fluorescence of the fluorogenic probe;
culturing the isolated cell to produce a first cell culture;
repeating the previous steps to isolate a second cell culture;
comparing the maximum-attained density of the first and second cell cultures; and
identifying the cell culture with increased cell density wherein cells in the cell culture expresses high levels of the RNA of interest.

21. The method of claim 20, wherein detection of fluorescence is carried out using flow cytometric cell sorting technology.

22. The method of claim 20, wherein said cell is a mammalian, bacterial insect, plant, algal or fungal cell.

23. The method of claim 22, wherein said mammalian cell is selected from the group consisting of: a Chinese Hamster Ovary (CHO) cell, a NS0 cell, a HEK 293 cell, and a Per.C6 cell.

24. The method of claim 22, wherein said bacterial cell is a BL21 cell.

25. The method of claim 22, wherein said fungal cell is selected from the group consisting of: a Chrysosporium cell, an Aspergillus cell, a Trichoderma cell, a Dictyostelium cell, a Candida cell, a Saccharomyces cell, a Schizosaccharomyces cell and a Penicillium cell.

26. The method of claim 22, wherein said insect cell is a SF9 cell or a SF21 cell.

27. The method of claim 20, further comprising the step of contacting the cell that exhibits increased fluorescence of the fluorogenic probe with a reagent for monitoring an apoptotic or pre-apoptotic marker.

28. The method of claim 27, wherein said reagent is Annexin-V or propidium iodide.

29. The method of claim 20, wherein the RNA of interest is selected from the group consisting of: a messenger RNA that encodes a protein, an antisense RNA molecule, a structural RNA, a ribosomal RNA, an hnRNA, and an snRNA.

30. The method of claim 29, wherein said messenger RNA encodes an immunoglobulin heavy chain, an immunoglobulin light chain, a single chain Fv, an Fab, Fab′, or (Fab′)₂ antibody fragment or an antigen binding fragment of an immunoglobulin.

31. The method of claim 20, wherein the RNA of interest is an endogenous RNA.

32. The method of claim 20, wherein the RNA of interest is a heterologous RNA.

33. A method for isolating a cell with a biphasic growth profile, wherein the cell has an increased rate of proliferation in a first portion of the growth profile, and wherein the cell has a decreased rate of proliferation in a second portion of the growth profile, comprising the steps of:
contacting a first population of cells with a fluorescent reagent for monitoring the rate of cell proliferation;
culturing said population of cells at low cell density,
isolating a cell from said population of cells that exhibits altered fluorescence of the fluorescent reagent in the first portion of the growth profile;
culturing the isolated cell to produce a second population of cells;
contacting said second population of cells with a fluorescent reagent for monitoring the rate of cell proliferation;
culturing said second population of cells at high cell density; and,
isolating a cell exhibiting an unaltered or slower rate of alteration of fluuorescence of the fluorescent reagent in the second portion of the growth profile as compared to the altered fluorescence of the fluorescenct reagent in the first portion of the growth profile, thereby isolating a cell with a biphasic growth profile, wherein the cell has an increased rate of proliferation in a first portion of the growth profile, and wherein the cell has a decreased rate of proliferation in a second portion of the growth profile.

34. A method for isolating a cell with a biphasic growth profile, wherein the cell has an increased rate of proliferation in a first portion of the growth profile, and wherein the cell has a decreased rate of proliferation in a second portion of the growth profile, and wherein the cell expresses high levels of an RNA of interest in the second portion of the growth profile, comprising the steps of:
contacting a population of cells with a fluorogenic probe that fluoresces upon hybridization to said RNA of interest;
contacting said population with a fluorescent reagent for monitoring the rate of cell proliferation, wherein the reagent fluoresces at a wavelength different than that of the fluorogenic probe; and
isolating a cell that exhibits altered fluorescence of the fluorescent reagent in the first portion of the growth profile, unaltered or decreased change of fluorescence of the fluorescent reagent in the second portion of the growth profile, and increased fluorescence of the fluorogenic probe in the second portion of the growth profile.

35. The method of claim 34, wherein the cell grows to increased density in the first portion of the growth profile.

36. The method of claim 34, wherein detection of the fluorescence of the fluorogenic probe is assayed simultaneously with detection of the fluorescence of the fluorescent reagent during the second portion of the growth profile.

37. The method of claim 34, wherein detection of fluorescence is carried out using flow cytometric cell sorting technology.

38. The method of claim 34, wherein said fluorescent reagent for monitoring the rate of cell proliferation is selected from the group consisting of: carboxyfluorescein diacetate succinimidyl ester and SNARF-1 carboxylic acid, acetate succinimidyl ester.

39. The method of claim 34, wherein said cell is a mammalian, bacterial, insect, plant, microbial, algal or fungal cell.

40. The method of claim 39, wherein said mammalian cell is selected from the group consisting of: a Chinese Hamster Ovary (CHO) cell, a NS0 cell, a HEK 293 cell, and a Per.C6 cell.

41. The method of claim 39, wherein said bacterial cell is a BL21 cell.

42. The method of claim 39, wherein said fungal cell is selected from the group consisting of: a Chrysosporium cell, an Aspergillus cell, a Trichoderma cell, a Dictyostelium cell, a Candida cell, a Saccharomyces cell, a Schizosaccharomyces cell and a Penicillium cell.

43. The method of claim 39, wherein said insect cell is a SF9 cell or a SF21 cell.

44. The method of claim 34, further comprising the step of contacting the cell that exhibits increased fluorescence of the fluorogenic probe and unaltered fluorescence of the fluorescent reagent with a reagent for monitoring an apoptotic or pre-apoptotic marker.

45. The method of claim 44, wherein said reagent is Annexin-V or propidium iodide.

46. The method of claim 34, wherein the RNA of interest is selected from the group consisting of: a messenger RNA that encodes a protein, an antisense RNA molecule, a structural RNA, a ribosomal RNA, an hnRNA, and an snRNA.

47. The method of claim 46, wherein said messenger RNA encodes an immunoglobulin heavy chain, an immunoglobulin light chain, a single chain Fv, an Fab, Fab′, or (Fab′)₂ antibody fragment, or an antigen binding fragment of an immunoglobulin.

48. The method of claim 34, wherein the RNA of interest is an endogenous RNA.

49. The method of claim 34, wherein the RNA of interest is a heterologous RNA.

50. The method of claim 34, further comprising the step of culturing the isolated cell to produce a cell culture.

51. The method of claim 50, further comprising the step of measuring the density of the cell culture.
